(19) United States
(12) Patent Application Publication
(10) Pub. No.: US 2015/0292012 A1
(43) Pub. Date: Oct. 15, 2015

(54) BIOMARKERS FOR NOD2 AND/OR RIP2 ACTIVITY RELATED APPLICATION

(71) Applicant: CASE WESTERN RESERVE UNIVERSITY, Cleveland, OH (US)

(72) Inventors: Derek Abbott, Cleveland, OH (US); Justine Tigno-Aranjuez, Cleveland, OH (US)

(21) Appl. No.: 14/438,797

(22) PCT Filed: Oct. 28, 2013
(86) PCT No.: PCT/US13/67089

§ 371 Date: Apr. 27, 2015

Related U.S. Application Data
(60) Provisional application No. 61/718,887, filed on Oct. 26, 2012.

(51) Int. Cl. C12Q 1/68; A61K 31/5377

(52) U.S. Cl. CPC C12Q 1/6883; A61K 31/5377; C12Q 2600/158; C12Q 2600/118; C12Q 2600/16

(57) ABSTRACT

A method of predicting RIP2 inhibitor efficacy in treating a subject with an inflammatory disorder includes obtaining a bodily sample from the subject, determining in the bodily sample the expression level(s) of at least one gene selected from the group consisting of cd40, Clec4E, clec5a, CxCL10, gpr84, Icam1, Irgl, Marcks11, pde4b, Ptges, Rasgrp1, and slc2a6,comparing the expression levels of the at least one gene with the corresponding control value(s), and characterizing the subject as being responsive to RIP2 inhibitor treatment if the expression levels of the at least one gene is increased compared to the corresponding control value(s).

### RELATED APPLICATION

This application claims priority from U.S. Provisional Application No. 61/718,887, filed Oct. 26, 2012, the subject matter of which is incorporated herein by reference in its entirety.

### BACKGROUND

Lack of coordination between inflammatory signaling pathways influences the development of inflammatory disorders, such as sacrcoidosis, rheumatoid arthritis, and inflammatory bowel disease. Inflammatory signal coordination can be modeled through the study of NLRP protein, NOD2. NOD2 was originally identified as the first Crohn's disease susceptibility gene. In the years since that discovery, NOD2 has been genetically linked to other inflammatory diseases, such as Blau Syndrome and Early Onset Sarcoidosis (EOS).

Crohn's disease affects 1 in 500/1000 Americans (approximately 440,000 people), and sarcoidosis affects approximately 154,000 Americans with the majority being African American. The card15 gene (coding for NOD2) is the most prevalent genetic polymorphism/mutation encountered in either of these patient populations.

Treatment for both of these disorders currently relies on broad, non-specific immunologic inhibition (e.g., corticosteroids) or on specific cytokine inhibition (e.g., anti-TNF therapies) with significant costs and side effects. Treatment is less than ideal, however, because not all agents are equally efficacious, the diseases occur over long time frames, and not all agents remain efficacious in the same patient.

### SUMMARY

Embodiments described herein relate to biomarkers associated with nucleotide-binding oligomerization domain containing 2 (NOD2) driven or mediated inflammatory disorders and/or immunological disorders and/or associated with high RIP2 kinase activity.

In some embodiments, the biomarkers can be used in methods of predicting RIP2 inhibitor efficacy in treating a subject with an inflammatory disorder and/or immunological disorder. The methods can include obtaining a biological sample from the subject. The expression level(s) of at least one gene selected from the group consisting of cd40, Clec4E, clec5a, CxCL10, gpr84, Icam1, Irg1, Marcks11, pde4b, Ptges, Rasgrp1, and slc2a6 is then determined in the biological sample. The expression level(s) of the at least one gene in the biological sample is compared with a corresponding control value(s). The subject is characterized as being responsive to RIP2 inhibitor treatment if the expression level(s) of the at least one gene is increased compared to the corresponding control value(s).

In some embodiments, the expression levels of at least two, three, four, five, six, seven, eight, nine, ten, eleven, or twelve genes selected from the selected from the group consisting of cd40, Clec4E, clec5a, CxCL10, gpr84, Icam1, Irgl, Marcks11, pde4b, Ptges, Rasgrp1, and slc2a6 is determined and compared with corresponding control values. The subject is then characterized as being responsive to RIP2 inhibitor treatment if the expression levels of the at least two, three, four, five, six, seven, eight, nine, ten, eleven, or twelve genes are increased compared to the corresponding control values. For example, the subject can be characterized as being responsive to RIP2 inhibitor treatment if the expression levels of the at least one, two, three, four, five, six, seven, eight, nine, ten, eleven, or twelve genes are increased at least 5, 10, 15, 20, 30, 40, or 50 fold compared to the corresponding control value(s).

In other embodiments, the inflammatory disease and/or immunological disorder can be associated with muramyl dipeptide (MDP)-induced, NFκB activation. The inflammatory disease can be selected from the group consisting of sacroidosis, rheumatoid arthritis, Crohn's disease, Blau syndrome, early onset sarcoidosis, colitis, asthma, graft versus host disease, and inflammatory bowel disease.

In some embodiments, the expression level of the at least one gene can be measured by measuring RNA level(s) corresponding to the at least one gene in the biological sample by, example, RNA sequencing using quantitative polymerase chain reaction to measure the RNA levels in the bodily sample.

In other embodiments, the biological sample can include at least one of peripheral blood mononuclear cells, monocytes, macrophages, epithelial cells, or cells of inflamed tissue that have been isolated from the subject. For instance, the biological sample can include cells from the intestinal lamina propia of a subject having or suspected of having sacroidosis, Crohn's disease, Blau syndrome, early onset sarcoidosis, colitis, or inflammatory bowel disease.

Other embodiments described herein relate to the use of the biomarkers in methods of monitoring the responsiveness of a subject with an inflammatory disorder and/or immunological disorder associated with nucleotide-binding oligomerization domain containing 2 (NOD2) activation to treatment with a RIP2 inhibitor. The methods can include administering to the subject a therapeutically effective amount of at least one RIP2 inhibitor. A biological sample is obtained from the subject after administration of the RIP2 inhibitor. The expression level(s) of at least one gene selected from the group consisting of cd40, Clec4E, clec5a, CxCL10, gpr84, Icam1, Irgl, Marcks11, pde4b, Ptges, Rasgrp1, and slc2a6 is determined in the biological sample. The expression level(s) of the at least one gene is compared with the corresponding control value(s). The subject is characterized as being responsive to the RIP2 inhibitor treatment if the expression levels of the at least one gene is decreased compared to the corresponding control value(s).

Still other embodiments described herein relate to the use of the biomarkers in methods for treating a subject with an inflammatory disorder and/or immunological disorder associated with nucleotide-binding oligomerization domain containing 2 (NOD2) activation. The methods can include obtaining a biological sample from the subject. The expression level of at least one gene selected from the group consisting of cd40, Clec4E, clec5a, CxCL10, gpr84, Icam1, Irgl, Marcks11, pde4b, Ptges, Rasgrp1, and slc2a6 is determined The expression level(s) of the at least one gene is compared with the corresponding controls. The subject is then administered a therapeutically effective amount of at least one RIP2 inhibitor if the expression levels of the at least one gene is increased compared to the corresponding control value(s).

Other embodiments described herein relate to a microarray for predicting RIP2 inhibitor efficacy in treating a subject with an inflammatory disorder. The microarray includes at least 5 polynucleotide probes having polynucleotide sequences complementary to the polynucleotide sequence of the corresponding differentially expressed genes selected from the group consisting of cd40, Clec4E, clec5a, CxCL10, gpr84, Icam1, Irgl, Marcks11, pde4b, Ptges, Rasgrp1, and slc2a6. In some embodiments, the microarray can be provided in a kit for predicting RIP2 inhibitor efficacy in treating a subject with an inflammatory disorder along with corresponding controls for the differentially expressed genes and a package for the microarray and the controls.

### BRIEF DESCRIPTION OF DRAWINGS

FIG. 1 illustrates a schematic drawing showing a strategy to identify kinase activity dependent and kinase activity independent functions of RIP2.

FIG. 2 illustrates graphs showing qRT-PCR validation of 10 genes whose expression was found to be inhibited under conditions of kinase inhibition.

FIG. 3 illustrates images showing RIP2 inhibition limits sarcoid-like phenotype in ITCH^{-/-} mice.

FIG. 4 illustrates images showing ileitis in the SAMP mice can be reversed by treatment with a RIP2 inhibitor.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which this application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application.

The term “activity” with reference to nuclear factor kappa-light-chain-enhancer of activated B cells (NFκB) activity can refer to a cellular, biological, and/or therapeutic activity or function of NFκB. Examples of such activities can include, but are not limited to, signal transduction, interacting or associating with DNA or other binding partner(s) or cellular component (s), and modulating cellular responses to stimuli, such as stress, cytokines, free radicals, UV radiation, oxidized LDL, and bacterial or viral antigens.

The terms “complementary” and “substantially complementary” refer to the hybridization, base pairing, or duplex formation between nucleotides or nucleic acids, such as, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. For example, selective hybridization may occur when there is at least about 65% complementary over a stretch of at least 14 to 25 nucleotides, preferably at least about 75%, and more preferably at least about 90% complementary.

The term “fragment” refers to a sub-sequence of a nucleic acid that is of a sufficient size and confirmation to properly function as a hybridization probe, as a primer in a PCR, or in another manner characteristic of nucleic acids.

The term “hybridization” refers to the formation of a duplex structure by two single-stranded nucleic acids due to fully (100%) or less than fully (less than 100%) complementary base pairing. Hybridization can occur between fully and complementary nucleic acid strands, or between less than fully complementary nucleic acid strands which contain regions of mismatch due to one or more nucleotide substitutions, deletions, or additions.

The term “kit” refers to any delivery system for delivering materials or reagents for carrying out a method of the present invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits can include one or more enclosures (e.g., boxes) containing the relevant reaction reagents and/or supporting materials for assays of the present invention.

The term “oligonucleotide” refers to a linear polymer of nucleotide monomers. Monomers making up oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their inter-nucleosidic linkages may be naturally occurring or may be analogs thereof, e.g., naturally occurring or non-naturally occurring analogs. Non-naturally occurring analogs may include PNAs, phosphorothioate inter-nucleosidic linkages, bases containing linking groups permitting the attachment of labels, such as fluorophores, or haptens, and the like. Whenever the use of an oligonucleotide requires enzymatic processing, such as extension by a polymerase, ligation by a ligase, or the like, one of ordinary skill would understand that oligonucleotides in those instances would not contain certain analogs of inter-nucleosidic linkages, sugar moities, or bases at any or some positions.

The term “polynucleotide” can refer to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material natural or synthetic in origin, including, e.g., iRNA, siRNA, microRNA, ribonucleoproteins (e.g., iRNPs). The term can also encompass nucleic acids, i.e., oligonucleotides, containing known analogues of natural nucleotides. Additionally, the term can encompass nucleic acid-like structures with synthetic backbones. Polynucleotides typically range in size from a few monomeric units, e.g., 5-40, when they are usually referred to as “oligonucleotides,” to several thousand monomeric units. Whenever a polynucleotide or an oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context.

The term “PCR” refers to a reaction for the in vitro amplification of specific DNA sequences by the simultaneous primer extension of complementary strands of DNA. In other words, PCR is a reaction for making multiple copies or replicates of a target nucleotide sequence flanked by primer binding sites. PCR typically comprises one or more repetitions of the following steps: (i) denaturing a target nucleotide sequence; (ii) annealing primers to primer binding sites; and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art. For example, in a conventional PCR using Taq DNA polymerase, a double-stranded target nucleotide sequence may be denatured at a temperature >90° C., primers annealed at a temperature in the range 50-75° C., and primers extended at a temperature in the range 72-78° C. Reaction volumes range from a few hundred nanoliters, e.g., 200 nl, to a few hundred μl, e.g., 200 μl. The term “PCR” encompasses derivative forms of the reaction, including but not limited to, RT-PCR, real-time PCR, nested PCR, quantitative PCR, multiplexed PCR, and the like.

The term “reverse transcription PCR,” or “RT-PCR,” refers to a PCR that is preceded by a reverse transcription reaction that converts a target RNA to a complementary single stranded DNA, which is then amplified.

The term “real-time PCR” refers to a PCR for which the amount of reaction product is monitored as the reaction proceeds. There are many forms of real-time PCR that differ mainly in the detection chemistries used for monitoring the reaction product.

The term “nested PCR” refers to a two-stage PCR wherein the amplified product of a first PCR becomes the sample for a second PCR using a new set of primers, at least one of which binds to an interior location of the first reaction product. “Outer primers” in reference to a nested amplification reaction refer to the primers used to generate a first reaction product, and “inner primers” refer to the one or more primers used to generate a second, or nested, reaction product.

The term “multiplexed PCR” refers to a PCR wherein multiple target sequences (or a single target sequence and one or more reference sequences) are simultaneously carried out in the same reaction mixture. Usually, distinct sets of primers are employed for each sequence being amplified.

The term “quantitative PCR” refers to a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates.

The term “primer” refers to a polynucleotide or oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

The term “target nucleotide sequence” refers to a region of a nucleotide which is to be amplified, detected, or otherwise analyzed. An oligonucleotide primer hybridizes to a region of the polynucleotide template immediately flanking the target nucleotide sequence.

The terms “inflammatory disorder” or “inflammatory disease” can refer to a disorder or disease characterized by aberrant activation of the immune system that leads to or causes pathogenesis of several acute and chronic conditions including, for example, sarcoidosis, rheumatoid arthritis, inflammatory bowel disease, transplant rejection, colitis, gastritis and ileitis. An inflammatory disease can include a state in which there is a response to tissue damage, cell injury, an antigen, an infectious disease, and/or some unknown cause. Symptoms of inflammation may include, but are not limited to, cell infiltration and tissue swelling.

The term “subject” can refer to any animal, including, but not limited to, humans and non-human animals (e.g., rodents, arthropods, insects, fish), non-human primates, ovines, bovines, ruminants, lagomorphs, porcines, caprines, equines, canines, felines, and ayes.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The term “therapeutically effective amount” can refer to that amount of one or more agents (e.g., a tyrosine kinase inhibitor) that result in amelioration of inflammatory disease symptoms or a prolongation of survival in a subject. A therapeutically relevant effect relieves to some extent one or more symptoms of an inflammatory disease or returns to normal, either partially or completely, one or more physiological or biochemical parameters associated with or causative of the disease.

The term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The phrases “parenteral administration” and “administered parenterally” means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” mean the administration of a compound, drug or other material other than directly into the central nervous system, such that it enters the animal's system and, thus, is subject to metabolism and other like processes, for example, subcutaneous administration.

The term “diagnosis” refers to a process aimed at determining if an individual is afflicted with a disease or ailment.

The term “sample” refers to a quantity of material from a biological, medical, or subject source in which detection or measurement of target nucleotide sequence is sought. On the one hand, the term is meant to include a specimen or culture (e.g., microbiological cultures). On the other hand, it is meant to include biological samples.

The term “biological sample” is used herein in its broadest sense. A biological sample may be obtained from a subject (e.g., a human) or from components (e.g., tissues) of a subject. The sample may be of any biological tissue or fluid with which biomarkers of the present invention may be assayed. Frequently, the sample will be a “clinical sample”, i.e., a sample derived from a patient or “bodily sample”. Such samples include, but are not limited to, bodily fluids, which may or may not contain cells, e.g., blood, tissue, or biopsy samples, such as from the intestines or lungs; and archival samples with known diagnosis, treatment and/or outcome history. Biological samples may also include sections of tissues, such as frozen sections taken from histological purposes. The term biological sample also encompasses any material derived by processing the biological sample. Derived materials include, but are not limited to, cells (or their progeny) isolated from the sample, proteins or nucleic acid molecules extracted from the sample. Processing of the biological sample may involve one or more of: filtration, distillation, extraction, concentration, inactivation of interfering components, addition of reagents, and the like.

The terms “normal' and “healthy” are used herein interchangeably. They refer to an individual or group of individuals who have not shown any inflammatory disease and/or immunological disorder symptoms. The normal individual (or group of individuals) can include those that are not on medication affecting inflammatory disease and/or immunological disorder and has not been diagnosed with any other disease. More preferably, normal individuals have similar sex, age, body mass index as compared with the individual from which the sample to be tested was obtained. The term “normal” is also used herein to qualify a sample isolated from a healthy individual.

The term “control sample” refers to one or more biological samples isolated from an individual or group of individuals that are normal (i.e., healthy). The term “control sample” (or “control” or “control value(s)”) can also refer to the compilation of data derived from samples of one or more individuals classified as normal, or one or more individuals diagnosed with an inflammatory disease and/or immunological disorder, or one or more individuals having undergone treatment of an inflammatory disease and/or immunological disorder.

The term “biomarker” refers to nucleic acid molecules comprising a nucleotide sequence which is expressed by a gene as well as polynucleotides that hybridize with portions of these nucleic acid molecules.

The term “indicative of NOD2 driven inflammatory disease and/or immunological disorder”, when applied to a biomarker, refers to an expression pattern or profile, which is diagnostic of the NOD2 driven inflammatory disease and/or immunological disorder or a stage of the NOD2 driven inflammatory disease and/or immunological disorder such that the expression pattern is found significantly more often in patients with the disease or a stage of the disease than in patients without the disease or another stage of the disease (as determined using routine statistical methods setting confidence levels at a minimum of 95%). Preferably, an expression pattern, which is indicative of NOD2 driven inflammatory disease and/or immunological disorder is found in at least 60% of patients who have the disease and is found in less than 10% of subjects who do not have the disease. More preferably, an expression pattern which is indicative of NOD2 driven inflammatory disease and/or immunological disorder is found in at least 70%, at least 75%, at least 80%, at least 85%, at least 90%, at least 95% or more in patients who have the disease and is found in less than 10%, less than 8%, less than 5%, less than 2.5%, or less than 1% of subjects who do not have the disease.

The term “differentially expressed biomarker” refers to a biomarker whose level of expression is different in a subject (or a population of subjects) afflicted with the NOD2 driven inflammatory disease and/or immunological disorder relative to its level of expression in a healthy or normal subject (or a population of healthy or normal subjects). The term also encompasses a biomarker whose level of expression is different at different stages of the disease. Differential expression includes quantitative, as well as qualitative, differences in the temporal or cellular expression pattern of the biomarker. As described in greater details below, a differentially expressed biomarker, alone or in combination with other differentially expressed biomarkers, is useful in a variety of different applications in diagnostic, staging, therapeutic, drug development and related areas. The expression patterns of the differentially expressed biomarkers disclosed herein can be described as a fingerprint or a signature of NOD2 driven inflammatory disease and/or immunological disorder progression. They can be used as a point of reference to compare and characterize unknown samples and samples for which further information is sought. The term “decreased level of expression”, as used herein, refers to a decrease in expression of at least 10% or more, for example, 20%, 30%, 40%, or 50%, 60%, 70%, 80%, 90% or more, or a decrease in expression of greater than 1-fold, 2-fold, 3-fold, 4-fold, 5-fold, 10-fold, 50-fold, 100-fold or more as measured by one or more methods described herein. The term “increased level of expression”, as used herein, refers to an increase in expression of at least 10% or more, for example, 20%, 30%, 40%, or 50%, 60%, 70%, 80%, 90% or more or an increase in expression of greater than 1-fold, 2-fold, 3fold, 4-fold, 5-fold, 10-fold, 50-fold, 100-fold or more as measured by one or more methods described herein.

As used herein, the term “a reagent that specifically detects expression levels” refers to one or more reagents used to detect the expression level of one or more biomarkers (e.g., a polynucleotide that hybridizes with at least a portion of the nucleic acid molecule). Examples of suitable reagents include, but are not limited to, nucleic acid probes capable of specifically hybridizing to a polynucleotide sequence of interest, or PCR primers capable of specifically amplifying a polynucleotide sequence of interest. The term “amplify” is used herein in the broad sense to mean creating/generating an amplification product. “Amplification”, as used herein, generally refers to the process of producing multiple copies of a desired sequence, particularly those of a sample. A “copy” does not necessarily mean perfect sequence complementarity or identity to the template sequence.

The terms “array”, “micro-array”, and “biochip” are used herein interchangeably. They refer to an arrangement, on a substrate surface, of hybridizable array elements, preferably, multiple nucleic acid molecules of known sequences. Each nucleic acid molecule is immobilized to a discrete spot (i.e., a defined location or assigned position) on the substrate surface. The term “micro-array” more specifically refers to an array that is miniaturized so as to require microscopic examination for visual evaluation.

The term “probe”, as used herein, refers to a nucleic acid molecule of known sequence, which can be a short DNA sequence (i.e., an oligonucleotide), a PCR product, or mRNA isolate. Probes are specific DNA sequences to which nucleic acid fragments from a test sample are hybridized. Probes specifically bind to nucleic acids of complementary or substantially complementary sequence through one or more types of chemical bonds, usually through hydrogen bond formation.

The terms “labeled”, “labeled with a detectable agent” and “labeled with a detectable moiety” are used herein interchangeably. These terms are used to specify that an entity (e.g., a probe) can be visualized, for example, following binding to another entity (e.g., a polynucleotide or polypeptide). Preferably, the detectable agent or moiety is selected such that it generates a signal which can be measured and whose intensity is related to the amount of bound entity. In array-based methods, the detectable agent or moiety is also preferably selected such that it generates a localized signal, thereby allowing spatial resolution of the signal from each spot on the array. Methods for labeling polypeptides or polynucleotides are well-known in the art. Labeled polypeptides or polynucleotides can be prepared by incorporation of or conjugation to a label, that is directly or indirectly detectable by spectroscopic, photochemical, biochemical, immunochemical, electrical, optical, or chemical means. Suitable detectable agents include, but are not limited to, various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

The term “computer readable medium” refers to any device or system for storing or providing information (e.g., data and instructions) to a computer processor. Examples of computer readable media include, but are not limited to, DVDs, CDs, hard disk drives, magnetic tape and servers for streaming media over networks.

Embodiments described herein relate to biomarkers associated with nucleotide-binding oligomerization domain containing 2 (NOD2) driven or mediated inflammatory disorders and/or immunological disorders and/or associated with high RIP2 kinase activity. It was previously found that tyrosine kinase inhibitors, which can inhibit RIP2 kinase activity, can dampen or inhibit NOD2:RIP2 signaling complex activation of NKκB and other pathways downstream of NOD2:RIP2 and be used to treat inflammatory disorders and/or immunological disorders in which NOD2 is active. As not all inflammatory diseases and/or immunological disorders are NOD2 driven or associated with high RIP2 kinase activity, it is desirable to identify subjects with inflammatory diseases and/or immunological disorders who might respond to RIP2 inhibition and/or in whom RIP2 inhibition can be especially efficacious. This can avoid unnecessary toxicity in subjects with inflammatory diseases and/or immunological disorders that are not NOD2 driven or have low RIP2 activity.

As shown in the Example, we identified NOD2:RIP2 regulated differentially expressed genes whose expression can predict RIP2 inhibition efficacy in a subject with an inflammatory disease and/or immunological disorder using RNA-seq to detect MDP-induced genes commonly inhibited by RIP2 kinase inhibitors. The differentially expressed genes are listed in Table 1 of the Example and include cd40, Clec4E, clec5a, CxCL10, gpr84, Icam1, Irgl, Marcks11, pde4b, Ptges, Rasgrp1, and slc2a6. It was found that all of these genes are upregulated at least 5 fold, 10 fold, 15 fold, 20 fold, 30 fold, 40 fold, 50 fold or more in bone marrow derived macrophages (BMDMs) upon MDP treatment, and their expression decreases substantially upon low-dose RIP2 kinase inhibition. Advantageously, as a group these genes' upregulation was specific to NOD2.

In some embodiments, the differentially expressed genes can be used as biomarkers in methods of predicting RIP2 inhibitor efficacy in treating a subject with an inflammatory disorder and/or immunological disorder. The methods can include obtaining a biological sample from the subject, determining the expression levels of at least one, two, three, four, five, six, seven, eight, nine, ten, eleven or twelve genes selected from the selected from the group consisting of cd40, Clec4E, clec5a, CxCL10, gpr84, Icam1, Irgl, Marcks11, pde4b, Ptges, Rasgrp1, and slc2a6, and comparing the determined expression level(s) with corresponding control value(s). The subject is then characterized as being responsive to RIP2 inhibitor treatment if the expression level(s) of the at least one, two, three, four, five, six, seven, eight, nine, ten, eleven, or twelve genes is increased compared to the corresponding control value(s). For example, the subject can be characterized as being responsive to RIP2 inhibitor treatment if the expression level of the at least one gene is increased at least 5, 10, 15, 20, 30, 40, or 50 fold compared to the corresponding control value(s).

Generally, the inflammatory disorder and/or immunological disorder can include any condition, disease, or disorder where the NFκB signal transduction pathway and/or NFκB activity in a cell of the subject can be modulated (e.g., decreased or inhibited) and/or where the inflammatory disorder results from other pathways downstream of NOD2:RIP2. Examples of cells in which the NFκB signal transduction pathway and/or NFκB activity can be modulated include immune cells, such as leukocytes, monocytes, and macrophages.

In some embodiments, the inflammatory disorder and/or immunological disorder can be selected from the group consisting of achlorhydra autoimmune active chronic hepatitis, acute disseminated encephalomyelitis, acute hemorrhagic leukoencephalitis, Addison's disease, agammaglobulinemia, alopecia areata, Alzheimer's disease, amyotrophic lateral sclerosis, ankylosing spondylitis, anti-gbm/tbm nephritis, antiphospholipid syndrome, antisynthetase syndrome, aplastic anemia, arthritis, asthma, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, halo disease/balo concentric sclerosis, Bechets syndrome, Berger's disease, Bickerstaff's encephalitis, Blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, colitis, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, graft versus host disease, Graves' disease, Guillain-barré syndrome (GB S), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (IBS), Kawasaki's disease, lichen planus, Lou Gehrig's disease, lupoid hepatitis, lupus erythematosus, ménière's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjogren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

In other embodiments, the inflammatory disease can include any condition, disease, or disorder associated with bacterial breakdown product-induced, NFκB activation. Examples of bacterial breakdown products can include MDP and lipopolysaccharide (LPS). Inflammatory disorders associated with MDP-induced, NFκB activation can include, for example, sarcoidosis (e.g., Early Onset Sarcoidosis or EOS), Blau Syndrome, inflammatory bowel disease (IBD) (e.g., Crohn's disease and ulcerative colitis), rheumatoid arthritis, colitis, gastritis, ileitis, asthma, and/or graft versus host disease.

The biological sample, which is obtained from the subject, can include any biologic or bodily sample from the subject in which the product of the differentially expressed genes (e.g., nucleic acid, such as mRNA) can be detected. The biological sample can include, for example, at least one of peripheral blood mononuclear cells, monocytes, macrophages, epithelial cells, or cells of inflamed tissue that have been isolated from the subject. In other examples, the biological sample can include cells from the intestinal lamina propia of a subject having or suspected of having sacroidosis, Crohn's disease, Blau syndrome, early onset sarcoidosis, colitis, and inflammatory bowel disease.

The biological samples used in the practice of the methods described herein may be fresh or frozen samples collected from a subject, or archival samples with known diagnosis, treatment and/or outcome history. Biological samples may be collected by any non-invasive means, such as by drawing blood from a subject, or using fine needle aspiration or needle biopsy. Alternatively, biological samples may be collected by an invasive method, including, for example, surgical biopsy.

In certain embodiments, the inventive methods are performed on the biological sample itself without or with limited processing of the sample.

In other embodiments, the inventive methods are performed at the single cell level (e.g., isolation of cells from the biological sample). However, in such embodiments, the inventive methods are preferably performed using a sample comprising many cells, where the assay is “averaging” expression over the entire collection of cells present in the sample. Preferably, there is enough of the biological sample to accurately and reliably determine the expression of the set of biomarkers of interest. Multiple biological samples may be taken from the same tissue/body part in order to obtain a representative sampling of the tissue.

In some embodiments, the methods described herein are performed on nucleic acid molecules extracted from the biological sample. For example, RNA may be extracted from the sample before analysis. Methods of RNA extraction are well known in the art (see, for example, J. Sambrook et al., “Molecular Cloning: A Laboratory Manual”, 1989, 2nd Ed., Cold Spring, Harbor Laboratory Press: Cold Spring Harbor, NY). Most methods of RNA isolation from bodily fluids or tissues are based on the disruption of the tissue in the presence of protein denaturants to quickly and effectively inactivate RNases. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.).

In certain embodiments, after extraction, mRNA is amplified, and transcribed into cDNA, which can then serve as template for multiple rounds of transcription by the appropriate RNA polymerase. Amplification methods are well known in the art (see, for example, A.R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNA polymerases (such as avian myeloblastosis virus reverse transcriptase or Moloney murine leukemia virus reverse transcriptase).

The method for determining the expression levels of the genes is not particularly limited, and all the gene detection methods known to those skilled in the art may be used. In some embodiments, the expression level of the at least one gene can be measured by measuring RNA level(s) corresponding to the at least one gene in the bological sample. Determination of expression levels of nucleic acid molecules in the practice of the methods described herein may be performed by any suitable method, including, but not limited to polymerase chain reaction (PCR) (see, for example, U.S. Pat Nos., 4,683,195; 4,683,202, and 6,040,166; “PCR Protocols: A Guide to Methods and Applications”, Innis et al. (Eds.), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991,88: 7276-7280), differential display (see, for example, Liang et al., Nucl. Acid. Res., 1993,21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, RAPID-SCAN, and the like.

Nucleic acid probes for use in the detection of polynucleotide sequences in biological samples may be constructed using conventional methods known in the art. Suitable probes may be based on nucleic acid sequences encoding at least 5 sequential amino acids from regions of nucleic acids encoding a protein marker, and preferably comprise 15 to 40 nucleotides. A nucleic acid probe may be labeled with a detectable moiety. The association between the nucleic acid probe and detectable moiety can be covalent or non-covalent. Detectable moieties can be attached directly to the nucleic acid probes or indirectly through a linker (E.S. Mansfield et al., Mol. Cell. Probes, 1995,9: 145-156). Methods for labeling nucleic acid molecules are well-known in the art (for a review of labeling protocols, label detection techniques and recent developments in the field, see, for example, L.J. Kricka, Ann. Clin. Biochem. 2002,39: 114-129; R.P. van Gijlswijk et al., Expert Rev. Mol. Diagn. 2001, 1: 81-91; and S. Joos et al., J. Biotechnol. 1994,35: 135-153).

Nucleic acid probes may be used in hybridization techniques to detect polynucleotides expressed by the genes. The technique generally involves contacting and incubating nucleic acid molecules isolated from a biological sample obtained from a subject with the nucleic acid probes under conditions such that specific hybridization can take place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of nucleic acid molecules comprising polynucleotide sequences of an expressed gene may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods described generally have at least 60%, preferably at least 75% and more preferably at least 90% identity to a portion of nucleic acids of the expressed gene.

Hybridization and amplification techniques described herein may be used to assay qualitative and quantitative aspects of expression of nucleic acid molecules comprising polynucleotide sequences of the expressed genes.

Alternatively, oligonucleotides or longer fragments derived from nucleic acids of each expressed gene may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et at., Science, 1995,270: 467-470; M. Schena et at., Proc. Natl. Acad. Sci. USA 1996, 93: 10614-10619; J,J. Chen et at., Genomics, 1998, 51: 313-324; U.S. Pat. Nos. 5,143,854; 5,445,934; 5,807,522; 5,837,832; 6,040,138; 6,045,996; 6,284,460; and 6,607,885).

Once the expression levels of the genes of interest have been determined (as described above) for the biological sample being analyzed, they are compared to the expression levels in one or more control samples or to at least one expression profile map for RIP2 kinase activity.

Comparison of expression levels according to methods described herein can be performed after the expression levels obtained have been corrected for both differences in the amount of sample assayed and variability in the quality of the sample used (e.g., amount and quality of mRNA tested). Correction may be carried out using different methods well-known in the art. For example, in samples containing nucleic acid molecules, correction may be carried out by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

The extent of the difference between the levels of the differentially expressed genes and their corresponding control values can be used to characterize the subject as being responsive to RIP2 inhibitor treatment. For example, if the expression levels of the at least one or more gene is increased at least 5, 10, 15, 20, 30, 40, or 50 fold compared to the corresponding control value(s) the subject will be responsive to RIP2 inhibitor treatment.

In some embodiments, comparison of each of the levels of the differentially expressed genes with a corresponding control value will provide difference value (e.g., fold change) for the particular differentially expressed gene being evaluated. By combining the difference values for a number of differentially expressed genes, one can obtain genetic profile score. Because the genetic profile score includes the differences of a number of different differentially expressed genes, it can provide a more accurate method for identifying whether a subject is responsive to RIP2 inhibition.

In some embodiments, control values are based upon the level of the differentially expressed genes in comparable biological samples obtained from a reference cohort. In some embodiments, the reference cohort can be a select population of human subjects. The control value is preferably provided in a manner that facilitates comparison with the level of the differentially expressed genes. In other words, it is preferable that the units used to represent the level of differentially expressed genes, if units are present, are the same units used for the control values. For example, it may be preferable to normalize the control values with the levels of expression of the corresponding differentially expressed genes. By “corresponding,” what is meant is that each differentially expressed gene has a “corresponding” control value for the same gene.

“Normalization” refers to statistical normalization. For example, according to one embodiment, a normalization algorithm is the process that translates the raw data for a set of microarrays into measure of concentration in each sample. A survey of methods for normalization is found in Sarkar et al., Nucleic Acids Res., 37(2), e17 (2009). For example, a microarray chip assesses the amount of mRNA in a sample for each of tens of thousands of genes. The total amount of mRNA depends both on how large the sample is and how aggressively the gene is being expressed. To compare the relative aggressiveness of a gene across multiple samples requires establishing a common baseline across the samples. Normalization allows one, for example, to measure concentrations of mRNA rather than merely raw amounts of mRNA.

The control value can take a variety of forms. The control value can be a single cut-off value, such as a median or mean. Corresponding control values for the expression level of differentially expressed genes can include, for example, mean levels, median levels, or “cut-off” levels, that are established by assaying a large sample of individuals and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., the disclosure of which is incorporated herein by reference. A “cutoff” value can be determined for each differentially expressed gene that is assayed.

In some embodiments, a predetermined value is used. A predetermined value can be based on the levels of differential gene expression in a biological sample taken from a subject at an earlier time. Unlike control values, predetermined values can be individualistic and need not be based on sampling of a population of subjects.

In some embodiments, it may be preferable to also include a system (e.g., computer system and/or software) that is configured to receive data related to the expression levels of differentially expressed genes, and optionally other patient data (e.g., related to other staging information) and to calculate and display a risk score. In some such embodiments, the system employs one or more algorithms to convert the data into a risk score. In some embodiments, the system comprises a database that associates differentially expressed gene levels with risk profiles, based, for example, on historic patient data, one or more control subjects, population averages, or the like. In some embodiments, the system comprises a user interface that permits a user to manage the nature of the information assessed and the manner in which the risk score is displayed. In some embodiments, the system comprises a display that displays a risk score to the user.

Further, in one embodiment, the computer program is also capable of normalizing the patient's gene expression levels in view of a standard or control prior to comparison of the patient's gene expression levels to those of the patient population. In some embodiments, the computer is capable of ascertaining raw data of a patient's expression values from, for example, RT-PCR or a microarray, or, in another embodiment, the raw data is input into the computer.

Other embodiments described herein relate to the use of the biomarkers in methods of monitoring the responsiveness of a subject with an inflammatory disorder and/or immunological disorder associated with nucleotide-binding oligomerization domain containing 2 (NOD2) activation to treatment with a RIP2 inhibitor.

The methods can include administering to the subject a therapeutically effective amount of at least one RIP2 inhibitor. The agent administered to the subject with the inflammatory and/or immunological disorder can include a small molecule, polypeptide, polynucleotide, other therapeutic composition, or combination thereof that is capable of decreasing or inhibiting phosphorylation of RIP2, RIP2 kinase activity, NOD2:RIP2 signaling, and/or NOD2:RIP2 complex activation of NFκB and other pathways downstream of NOD2:RIP2 in the NOD2-bearing cell without being cytoxic to the cell at therapeutically effective amounts. In one aspect, the agent can include a small molecule, polypeptide, polynucleotide, other therapeutic composition, or combination thereof which is capable of inhibiting phosphorylation of RIP2 (e.g., by inhibiting phosphorylation of Y474 RIP2). By inhibiting phosphorylation of RIP2, it is meant reducing phosphorylation of RIP2 in a NOD2-bearing cell, such as a leukocyte, upon NOD2 activation by at least about 10%, at least about 20%, at least about 30%, at least about 40%, at least about 50%, at least about 60%, at least about 70%, at least about 80%, at least about 90%, or at least about 95% compared to an untreated NOD2 activated leukocyte.

In an aspect of the application, an agent that is capable of inhibiting phosphorylation of RIP2 can include a tyrosine kinase inhibitor that is capable of decreasing or inhibiting RIP2 kinase activity and/or phosphorylation of RIP2. For example, the tyrosine kinase inhibitor can include a small molecule, polypeptide, polynucleotide, other therapeutic composition, or combination thereof which is capable of inhibiting the activity of the RIP2 kinase responsible for phosphorylating Y474 RIP2. Alternatively, the tyrosine kinase inhibitor can include a small molecule, polypeptide, polynucleotide, other therapeutic composition, or combination thereof which is capable of interacting with RIP2 so as to block (e.g., sterically block) or hinder addition of a phosphate group to Y474 RIP2. In some embodiments, the tyrosine kinase inhibitor, when administered at a therapeutically effective amount to a NOD2-bearing cell of subject being treated, can substantially inhibit RIP2 kinase in the NOD2-bearing cell (e.g., macrophage) to which it is administered without being cytoxic to the cell.

In some embodiments, a tyrosine kinase inhibitor that inhibits RIP2 kinase activity can include an epidermal growth factor receptor (EGFR) inhibitor. By “EGFR inhibitor”, it is meant an agent that inhibits EGFR tyrosine kinase by binding to the adenosine triphosphate (ATP)-binding site of the enzyme. It was found that tyrosine kinase inhibitors that are effective at selectively inhibiting the kinase activity of EGFR are also effective at inhibiting RIP2 kinase activity and RIP2 autophosphorylation of Y474 of RIP2. In an aspect of the application, the EGFR inhibitor can substantially inhibit RIP2 kinase in the immune cells (e.g., macrophage) or epithelial cell (e.g., Colonic epithelial cell) to which it is administered at nanomolar concentrations (e.g., about 10 nm to about 500 nm) without being cytoxic to the cell. In another aspect, the EGFR inhibitor can be as effective or more effective at inhibiting RIP2 kinase activity as inhibiting EGFR kinase activity.

EGFR inhibitors that are capable of inhibiting RIP2 kinase activity can include erlotinib and/or gefitinib, which are commercially available from respectively Genentech and AstraZeneca under the tradenames Tarceva and Iressa. It was found that erlotinib and gefitinib can substantially inhibit RIP2 kinase in NOD2-bearing cells (e.g., macrophage or epithelial cells) to which it is administered at nanomolar concentrations (e.g., about 10 nm to about 500 nm) without being cytoxic to the cells.

The agent can be administered (e.g., systemically or parenterally) to the subject in a pharmaceutical composition at a therapeutically effective amount and for a period of time effective to deliver the agent to at least one NOD2-bearing cell (e.g., a macrophage) in which the NFκB signal transduction pathway, NFκB activity, and/or other pathways downstream of NOD2:RIP2 can be modulated. It will be appreciated that the at least one agent may additionally comprise a pharmaceutically acceptable carrier. Pharmaceutically acceptable carriers are known in the art, and may include any material or materials, which are not biologically or otherwise undesirable, i.e., the material may be incorporated or added into the agent without causing any undesirable biological effects or interacting in a deleterious manner with any of the other components of the composition. When the term “pharmaceutically acceptable” is used to refer to a pharmaceutical carrier, it can be implied that the carrier has met the required standards of toxicological and manufacturing testing or that it is included on the Inactive Ingredient Guide prepared by the U.S. Food and Drug administration.

Following administration of the agent, a biological sample is obtained from the subject. The expression level(s) of at least one gene selected from the group consisting of cd40, Clec4E, clec5a, CxCL10, gpr84, Icam1, Irg1, Marcks11, pde4b, Ptges, Rasgrp1, and slc2a6 is determined in the biological sample. The expression level(s) of the at least one gene are compared with corresponding control value(s). The subject is characterized as being responsive to the RIP2 inhibitor treatment if the expression levels of the at least one gene is decreased compared to the corresponding control value(s). In some embodiments, the control value(s) can be the expression level(s) of the at least one gene in a biological sample obtained from the subject prior to the administration of the RIP2 inhibitor.

Still other embodiments described herein relate to the use of the biomarkers in methods for treating a subject with an inflammatory disorder and/or immunological disorder associated with nucleotide-binding oligomerization domain containing 2 (NOD2) activation. The methods can include obtaining a biological sample from the subject. The expression level(s) of at least one gene selected from the group consisting of cd40, Clec4E, clec5a, CxCL10, gpr84, Icam1, Irgl, Marcks11, pde4b, Ptges, Rasgrp1, and slc2a6 is determined The expression level(s) of the at least one gene is compared with the corresponding controls. The subject is then administered a therapeutically effective amount of at least one RIP2 inhibitor if the expression levels of the at least one gene is increased compared to the corresponding control value(s). Alternatively, the subject is administered a therapeutically effective amount of anti-inflammatory agent that is not a RIP2 inhibitor if the expression level(s) of the at least one gene is either not increased on minimally increased compared to the corresponding control value(s).

The following example is included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE

In this Example we identified biomarkers that can identify patients with NOD2-driven inflammatory disease and/or high RIP2 kinase activity. The biomarkers can be used to guide clinical trials and identify patients likely to respond to RIP2 inhibition.

RNA-seq to Identify a Transcriptomic Signature that Drives NOD2:RIP2-Inflammatory Disease

Microarray studies on NOD2 have shown that depending on the statistical cutoff, only 40-60 genes are up or down-regulated 2-fold when exposed to MDP, and often times, these results are not 100% reproducible by qRT-PCR. In contrast, because one is sequencing over 200 million independent RNA species, RNA-seq allows direct, quantitative comparisons over a much broader linear range with a much greater sensitivity. Thus, in contrast to microarray, the same experiment performed using Next-Gen sequencing identified 1078 transcripts significantly altered by MDP. 18 of these were potential lncRNAs, 14 were potential alternative splice variants and 3 were potential RNA editing events. Ingenuity analysis showed that while there were expected signaling pathways altered (such as NF-κB and MAPK), NOD2 activation affected a number of other signaling pathways as well including IL-17 signaling, viral responses, reactive oxygen species production and LTβ signaling.

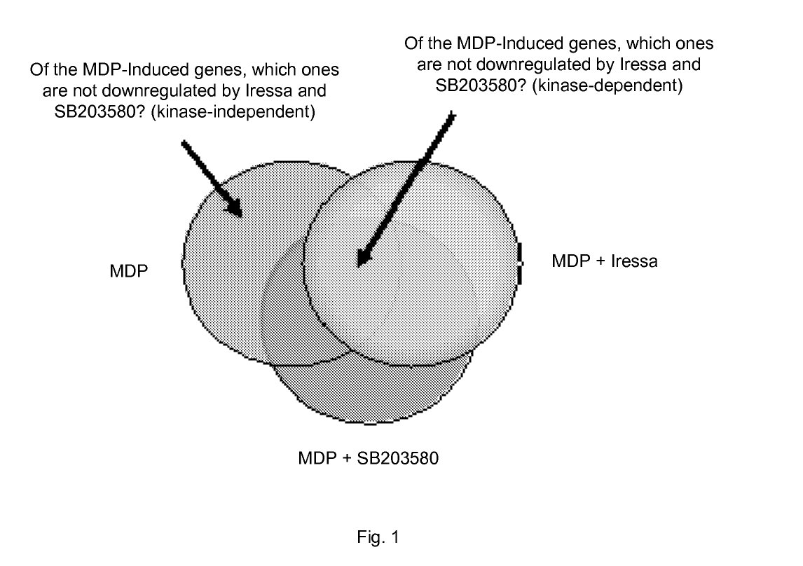

Given our successful use of RNA-seq, and given our goal of identifying biomarkers that could predict RIP2 inhibition efficacy in patients, we turned our attention to RIP2′s kinase activity. We initially discovered Tarceva and Iressa's inhibition of RIP2 as an off-target effect, and we further know from the literature and our own unpublished data that the p38 inhibitor, SB203580, inhibits RIP2 as well as it does p38. While Iressa and SB203580 inhibit other kinases in addition to RIP2, RIP2 is the only kinase that they inhibit in common. For this reason, we designed an RNA-seq experiment in which we either left BMDMs untreated or treated them with MDP, Iressa, SB203580, MDP+Iressa or MDP +SB203580 in duplicate (FIG. 1). Genes affected by drug alone were eliminated from the analysis. Although the bioinformatics is still ongoing, preliminary analysis indicates that approximately 25% of the genes upregulated by MDP are RIP2-kinase dependent while 75% are dependent on RIP2, but independent of RIP2's kinase activity. The MDP-induced genes that are commonly inhibited by Iressa and SB203580 are the MDP-induced genes that are likely to be dependent on RIP2′s kinase activity.

From this experiment, we identified a number of genes that are specifically affected by RIP2 inhibition. Of these transcripts, we focused on a core group of 12. This core group was chosen because they are all upregulated 10-50 fold upon MDP treatment, and they all decrease substantially upon low-dose RIP2 kinase inhibition. Most importantly, as a group these transcripts' upregulation was specific to NOD2. That is, bioinformatic analysis of gene expression databases showed that while individually, some of these genes were upregulated by inflammatory agonists like TNF or IL-1, collectively, this group is uniquely regulated by MDP. These transcripts are listed in Table 1.

TABLE 1

| | % inhibition | % inhibition |
|---|---|---|
| Gene | Iressa (200 nM) | SB203580 (200 nM) |
| cd40 | 55.6 | 46.3 |
| Clec4E | 67.4 | 69.8 |
| clec5a | 65.7 | 65.1 |
| CxCL10 | 70.0 | 65.5 |
| gpr84 | 68.4 | 68.9 |
| Icam1 | 47.4 | 43.0 |
| Irg1 | 59.7 | 54.8 |
| Marcksl1 | 59.9 | 61.9 |
| pde4b | 49.7 | 39.6 |
| Ptges | 62.6 | 64.6 |
| Rasgrp1 | 68.7 | 57.0 |
| slc2a6 | 45.7 | 46.0 |

To obtain the data in Table 1, primary BMDMs were treated for 30 minutes with 200 nM Iressa or 200 nM SB203580 before treatment with MDP. This minimal, 200 nM, amount of drug was used to eliminate potential artifacts of drug treatment. After 4 hours of MDP treatment, RNA was harvested and sequenced using an Illumina platform. Controls included Iressa treatment alone and SB203580 treatment alone. The experiment was done in duplicate using the following bioinformatic approach. The 100-bp paired-end Illumina reads were processed to remove the 3′ bases with Phred quality score of lower than 20. Reads that are less than 20 bases after quality trimming were removed from further analysis. The reads from each replicate of each sample were mapped to mouse genome release mm9 using tophat v1.4.1 program before guided assembly using cufflinks v1.3.0 program. Differential expression of transcripts was analyzed using twotailed Student t-test with Benjamini and Hochberg correction of false discovery rate (FDR). FDR corrected P value of 0.05 was set as the cutoff of statistical significance. The transcripts were then verified by qRT-PCR. While many potential RIP2 kinase dependent genes were identified, 12 of the most consistently down-regulated genes at this minimal dose of drug are shown.

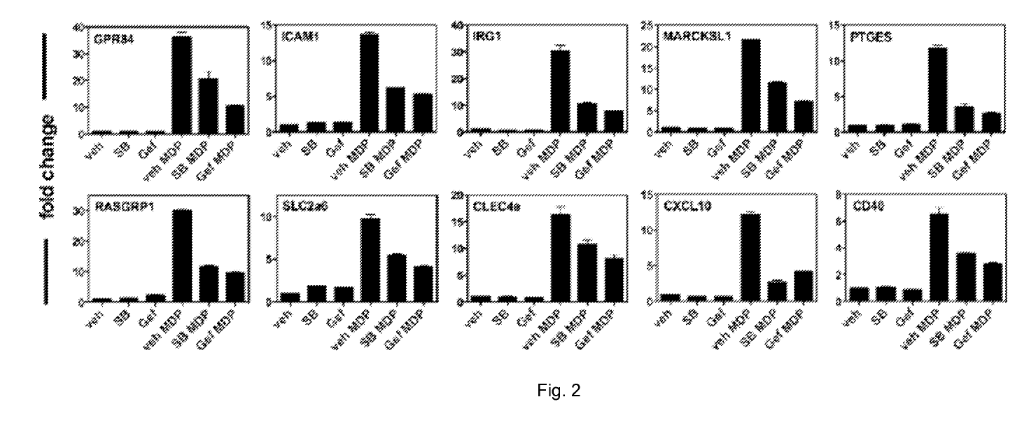

These genes' upregulation by MDP and inhibition by Iressa (Gefitinib) and SB203580 was verified by qRT-PCR (FIG. 2), and the RIP2 dependence was shown by a lack of induction in RIP2^{-/-} macrophages and by lack of induction using 2 RIP2 inhibitors from other companies.

We will verify that those genes whose expression is found to be altered as shown above are altered in mouse models of inflammatory disease. To this end, we use 3 models of inflammatory disease. We utilize the SAMP1/YitFc (SAMP) model of Crohn's-like ileitis. SAMP mice spontaneously develop a progressive, chronic ileitis that has clinical features similar to those observed in patients with Crohn's disease. SAMP mice have been found to be WT for Nodl and Nod2, and in my lab's hands, have normal NOD1 and NOD2 signaling and cytokine responses. Additionally, we also utilize a second inflammatory model to study IBD. IL-10^{-/-} mice develop a characteristic colitis at 10 weeks of age and are readily available from Jackson Labs Importantly, the NOD2^{-/-} mouse has been mated with the IL-10^{-/-} mouse and this double knockout mouse loses the chronic colitis. A last inflammatory model utilizes a sarcoidosis model of lung inflammation. In both IBD and sarcoidosis, genetic studies show that WT, hyperactive NOD2 can exacerbate inflammation. Data on the IBD and sarcoidosis models and the use of RIP2 inhibitors in these systems is described below. Briefly, to determine if genetic signatures in inflammatory models match those found by transcriptomics, ileal macrophages and intestinal epithelial cells are isolated from the lamina propria of IL-10^{-/-}, IL-10^{-/-}, SAMP or AKR control mice at four months of age. Cells are isolated from mouse bronchoalveolar lavage fluid (BALF) after sarcoidosis initiation. The genes identified above are evaluated by qRT-PCR and compared to RNA generated from either untreated sex-matched littermate mock-treated mice (sarcoidosis model), to control sex-matched parental AKR mice (ileitis model) or to IL-10^{-/-} or IL-10^{-/-} mice (colitis model). We anticipate that the genes that are found in Table 1 to be dependent on RIP2's kinase activity will be upregulated in NOD2-driven inflammatory disease.

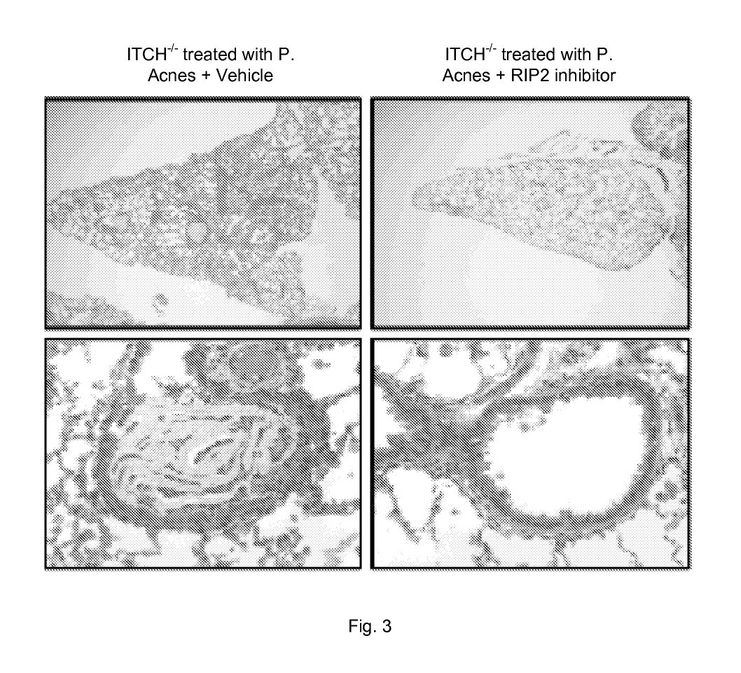

To determine the efficacy of RIP2 kinase inhibition in mouse models of inflammatory disease, we also utilize 3 different in vivo models of inflammatory disease (P. acnes: sarcoidosis, SAMP mice: ileitis, IL-10^{-/-} mice: ileocolitis). The first is a lung model that simulates sarcoidosis. Activating NOD2 mutations are responsible for an autosomal dominant form of sarcoidosis, and this scenario can be mimicked through the use of ITCH^{-/-} mice. We discovered that the E3 ubiquitin ligase ITCH directly ubiquitinates RIP2 to downregulate NOD2 signaling. ITCH^{-/-} mice have a hyperactive NOD2:RIP2 signaling pathway that manifests in increased MDP-induced cytokine responses. To determine if these mice are hypersensitive to sarcoidosis models, we utilized the P. acnes model of sarcoidosis. In this model, heat-killed P. acnes (an anaerobic bacterium often found in the granulomas of sarcoidosis patients) is injected intraperitoneally two weeks before intratracheal injection. After intratracheal injection, the mice develop granulomatous inflammation in their lungs that, in C57BL/6 mice, clears after 9 days. Because ITCH-mutant human patients all develop inflammatory lung disease and because loss of ITCH leads to hyperactivation of NOD2, we wanted to determine if the ITCH^{-/-} mice were hypersensitive to this sarcoidosis model. ITCH^{-/-} mice were subjected to the protocol and compared to sex-matched ITCH^{-/-} littermate controls. ITCH^{-/-} mice were significantly more sensitive to the sarcoidosis protocol. In fact, 3 days after P. acnes intratracheal injection, ITCH^{-/-} mice showed a marked increase in lung inflammation (FIG. 3). Pretreatment of the mice with Iressa (Gefitinib) reversed this pathology (FIG. 3).

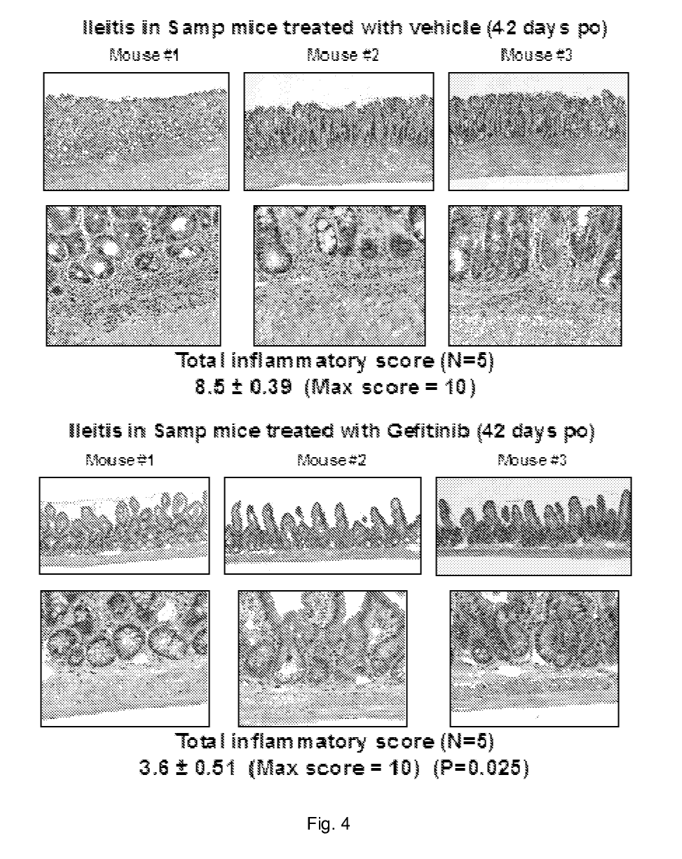

A second inflammatory disease model we utilize is the SAMP mouse model of Crohn's disease-like ileitis. Both the Nodl and Nod2 genes have been sequenced in SAMP mice and have been shown to be WT. While comparing between genetically distinct mouse strains is difficult, we see minimal differences in signaling or cytokine responses between the SAMP mice and AKR mice. To resolve this issue, we have mated the NOD2^{-/-} mice with the SAMP mice using speed congenics and see a dramatic decrease in SAMP ileitis suggesting that WT NOD2 activity helps cause ileitis in these mice. To further study this issue using a non-genetic approach, 6-wk-old SAMP mice were provided Gefitinib (Iressa) in their food supply at a daily dose of 10 mg/kg. Controls were sex-matched littermates treated with vehicle alone in their food supply. After 6 weeks of treatment, mice were sacrificed and Wei Xin, a GI Pathologist in our department, blindly analyzed the histology. In SAMP mice treated with vehicle only, there is very little villous height (FIG. 4), and this is an established pathologic marker of severe ileitis. In contrast, villous height is intact in the Gefitinib-treated mice (FIG. 4, lower panel). At the junction of the lamina propria and muscularis mucosa in the vehicle treated mice, there is a prominent band of inflammatory cells (neutrophils, macrophages and lymphocytes) characteristic of the acute inflammation in these mice (higher power, upper panels of FIG. 4). This is not seen in the Gefitinib-treated mice (lower panels, higher magnification, FIG. 4). There was a significant difference in inflammatory scores between the two groups of mice (FIG. 4). This experiment, coupled with the results of the NOD2^{-/-}:SAMP mouse mating, strongly suggests that RIP2 inhibition is efficacious in this model.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1-10. (canceled)

11. A method of monitoring the responsiveness of a subject with an inflammatory disorder and/or immunological disorder associated with nucleotide-binding oligomerization domain containing 2 (NOD2) activation to treatment with a RIP2 inhibitor, the method comprising:
administering to the subject a therapeutically effective amount of at least one RIP2 inhibitor;
obtaining a biological sample from the subject after administration of the RIP2 inhibitor,
determining in the biological sample the expression level(s) of at least one gene selected from the group consisting of cd40, Clec4E, clec5a, CxCL10, gpr84, Icam1, Irg1, Marcks11, pde4b, Ptges, Rasgrp1, and slc2a6,
comparing the expression level(s) of the at least one gene with corresponding control value(s), and
characterizing the subject as being responsive to the RIP2 inhibitor treatment if the expression level(s) of the at least one gene is decreased compared to the corresponding control value(s).

12. The method of claim 11, determining the expression levels of at least two genes selected from the selected from the group consisting of cd40, Clec4E, clec5a, CxCL10, gpr84, Icam1, Irg1, Marcks11, pde4b, Ptges, Rasgrp1, and slc2a6, comparing the expression levels of the at least two genes with the corresponding control values, and characterizing the subject as having an being responsive to the RIP2 inhibitor treatment if the expression levels of the at least two genes is decreased compared to the corresponding control values.

13. The method of claim 11, determining the expression levels of at least three genes selected from the selected from the group consisting of cd40, Clec4E, clec5a, CxCL10, gpr84, Icam1, Irg1, Marcks11, pde4b, Ptges, Rasgrp1, and slc2a6, comparing the expression levels of the at least three genes with the corresponding control values, and characterizing the subject as having an being responsive to RIP2 inhibitor treatment if the expression levels of the at least three genes is decreased compared to the corresponding control values.

14. The method of claim 11, the inflammatory disease being selected from the group consisting of sacroidosis, rheumatoid arthritis, Crohn's disease, Blau syndrome, early onset sarcoidosis, colitis, asthma, graft versus host disease, and inflammatory bowel disease.

15. The method of claim 11, measuring the expression level(s) of the at least one gene by measuring RNA level(s) of the corresponding at least one gene in the biological sample.

16. The method of claim 15, RNA sequencing using quantitative polymerase chain reaction to measure the RNA levels in the bodily sample.

17. The method of claim 11, wherein the biological sample comprises at least one of peripheral blood mononuclear cells, monocytes, macrophages, epithelial cells, or cells of inflamed tissue that have been isolated from the subject.

18. The method of claim 17, wherein the biological sample comprises cells from the intestinal lamina propia.

19. The method of claim 11, wherein the RIP2 inhibitor comprises at least one of erlotinib or gefitinib.

20. A method for treating a subject with an inflammatory disorder and/or immunological disorder associated with nucleotide-binding oligomerization domain containing 2 (NOD2) activation, the method comprising:
obtaining a biological sample from the subject after administration of the RIP2 inhibitor,
determining in the biological sample expression level(s) of at least one gene selected from the group consisting of cd40, Clec4E, clec5a, CxCL10, gpr84, Icam1, Irg1, Marcks11, pde4b, Ptges, Rasgrp1, and slc2a6,
comparing the expression level(s) of the at least one gene with corresponding control value(s), and
administering to the subject a therapeutically effective amount of at least one RIP2 inhibitor if the expression level(s) of the at least one gene is increased compared to the corresponding control value(s).

21. The method of claim 20, determining the expression levels of at least two genes selected from the selected from the group consisting of cd40, Clec4E, clec5a, CxCL10, gpr84, Icam1, Irg1, Marcks11, pde4b, Ptges, Rasgrp1, and slc2a6, comparing the expression level of the at least two genes with the corresponding control values, and characterizing the subject as having an being responsive to RIP2 inhibitor treatment if the expression levels of the at least two genes is increased compared to the corresponding control values.

22. The method of claim 20, determining the expression levels of at least three genes selected from the selected from the group consisting of cd40, Clec4E, clec5a, CxCL10, gpr84, Icam1, Irg1, Marcks11, pde4b, Ptges, Rasgrp1, and slc2a6, comparing the expression level of the at least three genes with the corresponding control values, and characterizing the subject as having an being responsive to RIP2 inhibitor treatment if the expression levels of the at least three genes is increased compared to the corresponding control values.

23. The method of claim 20, the inflammatory disease being selected from the group consisting of sacroidosis, rheumatoid arthritis, Crohn's disease, Blau syndrome, early onset sarcoidosis, colitis, asthma, graft versus host disease, and inflammatory bowel disease.

24. The method of claim 20, measuring the expression level(s) of the at least one gene by measuring RNA level(s) of the corresponding at least one gene in the biological sample.

25. The method of claim 24, RNA sequencing using quantitative polymerase chain reaction to measure the RNA levels in the biological sample.

26. The method of claim 20, wherein the biological sample comprises at least one of peripheral blood mononuclear cells, monocytes, macrophages, epithelial cells, or cells of inflamed tissue that have been isolated from the subject.

27. The method of claim 26, wherein the biological sample comprises cells from the intestinal lamina propia.

28. The method of claim 19, wherein the RIP2 inhibitor comprises at least one of erlotinib or gefitinib.

29-31. (canceled)
